(19) United States
(12) Patent Application Publication
(10) Pub. No.: US 2026/0062666 A1
(43) Pub. Date: Mar. 5, 2026

(54) METHOD FOR CULTURING COLD-RESISTANT NITRIFYING BACTERIA, METHOD FOR TREATING NITROGEN-CONTAINING WATER, AND DEVICE FOR TREATING NITROGEN-CONTAINING WATER

(71) Applicant: KUBOTA CORPORATION, Osaka (JP)

(72) Inventors: Shinichiro WAKAHARA, Amagasaki-shi (JP); Toshihiro KOMATSU, Amagasaki-shi (JP); Tatsuo SUMINO, Kawagoe-shi (JP); Qintong LI, Gunma (JP)

(21) Appl. No.: 19/106,738

(22) PCT Filed: Apr. 7, 2023
(86) PCT No.: PCT/JP2023/014416

§ 371 Date: Feb. 26, 2025

(30) Foreign Application Priority Data
Aug. 29, 2022 (JP) 2022-136167

(51) Int. Cl. C12N 1/20; C02F 3/12; C02F 101/16

(52) U.S. Cl. CPC C12N 1/20; C02F 3/1221; C02F 2101/16; C02F 2203/006

(57) ABSTRACT

Provided are (i) a method for culturing low temperature-resistant nitrifying bacteria by a simple method, (ii) a method for treating nitrogen-containing water with use of the low temperature-resistant nitrifying bacteria, and (iii) a device capable of culturing the low temperature-resistant nitrifying bacteria and treating nitrogen-containing water. A method for culturing low temperature-resistant nitrifying bacteria, the method including the step of: adjusting a pH of nitrogen-containing water a containing ammonia nitrogen and/or organic nitrogen to not more than 6.0 to obtain nitrogen-containing water b in which a dominant species of microorganisms is low temperature-resistant nitrifying bacteria.

### TECHNICAL FIELD

The present invention relates to a method for culturing low temperature-resistant nitrifying bacteria, a method for treating nitrogen-containing water, and a device for treating nitrogen-containing water.

### BACKGROUND ART

Biological treatment with use of nitrifying bacteria is used in treatment of ammonia nitrogen and/or organic nitrogen contained in sewage, industrial wastewater, or the like. Biological treatment is an inexpensive method but is difficult to conduct at low temperatures. That is, biological treatment has a problem that the reaction does not progress in a case where water to be treated, such as sewage, has a water temperature of not higher than 15° C., and it is therefore difficult to carry out the treatment in winter.

As such, conventionally, a method is employed in which heating equipment (steam blowing) is used to heat the water to be treated, and then the water is subjected to biological treatment. However, such a method is very cumbersome, and there is a demand for an alternative method.

A method disclosed in Patent Literature 1 can be named as an example of the alternative method. The method treats ammonia nitrogen-containing water at a low temperature of not higher than 15° C. with use of AH bacteria (Ammonia oxidizing bacteria detected by MPN method using High ammonium media) (MPN method: most probable number method), which are low temperature-resistant nitrifying bacteria.

### CITATION LIST

### Patent Literature

### [Patent Literature 1]

International Publication No. WO 2014/017429

### SUMMARY OF INVENTION

### Technical Problem

The method disclosed in Patent Literature 1 has already been put to practical use. However, in order to culture AH bacteria, it is necessary to use a large amount of a culture medium containing highly-concentrated ammonia. This makes culturing equipment enormous.

As such, there is a demand for a method for culturing low temperature-resistant nitrifying bacteria by a simpler method with use of simple equipment.

It is an object of an aspect of the present invention to provide (i) a method for culturing low temperature-resistant nitrifying bacteria by a simple method, (ii) a method for treating nitrogen-containing water with use of the low temperature-resistant nitrifying bacteria, and (iii) a device capable of culturing the low temperature-resistant nitrifying bacteria and treating nitrogen-containing water.

### Solution to Problem

In order to attain the object, a method in accordance with an aspect of the present invention for culturing low temperature-resistant nitrifying bacteria includes the step of: adjusting a pH of nitrogen-containing water a containing ammonia nitrogen and/or organic nitrogen to not more than 6.0 to obtain nitrogen-containing water b in which a dominant species of microorganisms is low temperature-resistant nitrifying bacteria.

A device in accordance with an aspect of the present invention for treating nitrogen-containing water is a device for treating nitrogen-containing water, including a culture tank and a nitrification tank, wherein:
the culture tank and the nitrification tank are capable of circulating content between each other;
the culture tank (i) is capable of storing therein (a) nitrogen-containing water a containing ammonia nitrogen and/or organic nitrogen and (b) return sludge obtained from the nitrification tank, and (ii) produces nitrogen-containing water b from the nitrogen-containing water a by adjusting a pH of the nitrogen-containing water a to not more than 6.0 and thereby causing a dominant species of microorganisms contained in the nitrogen-containing water a to be low temperature-resistant nitrifying bacteria; and
the nitrification tank (i) is capable of storing therein the nitrogen-containing water a and the nitrogen-containing water b and (ii) produces a nitrification liquid by nitrifying, at a water temperature of not lower than 0° C. and not higher than 15° C. with use of the low temperature-resistant nitrifying bacteria contained in the nitrogen-containing water b, ammonia contained in the nitrogen-containing water a and/or the nitrogen-containing water b.

A device in accordance with an aspect of the present invention for treating nitrogen-containing water is a device for treating nitrogen-containing water, the device including a treatment system A and a treatment system B, wherein:
the treatment system A includes a nitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the nitrification liquid obtained in the nitrification tank I, and a nitrification liquid obtained in the nitrification tank II, (ii) denitrifies nitric acid contained in each of the nitrification liquid obtained in the nitrification tank I and the nitrification liquid obtained in the nitrification tank II, and (iii) transfers the nitrogen-containing water a to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. in an aerobic atmosphere with use of the low temperature-resistant nitrifying bacteria, and (iii) transfers the nitrification liquid to the denitrification tank II.

Further, a device in accordance with an aspect of the present invention for treating nitrogen-containing water is a device for treating nitrogen-containing water, the device including a treatment system A and a treatment system B, wherein:
the treatment system A includes a nitrification tank I and a denitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the denitrification tank I denitrifies nitric acid contained in the nitrification liquid obtained in the nitrification tank I and transfers obtained return sludge to the treatment system B;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the return sludge from the denitrification tank I, and a nitrification liquid obtained in the nitrification tank II, (ii) denitrifies nitric acid contained in the nitrification liquid, and (iii) transfers the nitrogen-containing water a and the return sludge to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge, and (iii) transfers the nitrification liquid to the denitrification tank II.

### Advantageous Effects of Invention

According to an aspect of the present invention, it is possible to obtain low temperature-resistant nitrifying bacteria easily and in a large amount. That is, according to an aspect of the present invention, it is possible, without use of a large amount of a culture medium containing highly-concentrated ammonia, to cause a dominant species of microorganisms in nitrogen-containing water to be low temperature-resistant nitrifying bacteria, by a simple method of adjusting a pH of nitrogen-containing water.

Further, the low temperature-resistant nitrifying bacteria has a sufficient low-temperature resistance. As such, according to an aspect of the present invention, it is possible to carry out nitrification smoothly even in a case where the nitrogen-containing water has a low temperature of not higher than 15° C.

Further, according to an aspect of the present invention, it is possible to provide a device for treating nitrogen-containing water which has a simple structure and which is capable of culturing the low temperature-resistant nitrifying bacteria and treating nitrogen-containing water.

### BRIEF DESCRIPTION OF DRAWINGS

FIG. 1 is a view illustrating a result of nitrification carried out in Example 1 such that: a pH of the nitrogen-containing water was adjusted to 5; and ammonia contained in the nitrogen-containing water was nitrified at a temperature of 5° C. to 6° C. in an aerobic atmosphere.

FIG. 2 is a view illustrating the necessity of a bacterial count of 3×10⁶ copies/g in order to satisfy a nitrification rate of 0.05 kg-N/m³/day in a case where ammonia contained in nitrogen-containing water is nitrified at 5° C. by a method of causing comammox Nitrospira to be carried by a carrier (carrier method).

FIG. 3 is a view illustrating a result obtained in Example 4 by causing comammox Nitrospira to be carried by a carrier and studying growth properties of comammox Nitrospira at 25° C. with use of the carrier as seed bacteria.

FIG. 4 is a view illustrating a structure of a culture device used in Examples 5 etc. in order to adjust a pH of nitrogen-containing water to cause a dominant species of microorganisms to be comammox Nitrospira.

FIG. 5 is a view illustrating a result obtained in Example 5 by checking a change over time in quality of inorganic synthetic wastewater at 25° C. in an acidic environment.

FIG. 6 is a view illustrating a result obtained in Example 5 by carrying out real-time PCR of a bacterial colony carried by a carrier in inorganic synthetic wastewater having a temperature of 25° C. and a pH adjusted to 6, 5.5, and 5.

FIG. 7 is a view illustrating a result obtained in Example 6 by checking a change over time in quality of nitrogen-containing water having a temperature of 5° C. to 6° C. and a pH adjusted to 6.

FIG. 8 is a view illustrating a relationship between a volume load and a nitrification rate in the device illustrated in FIG. 4 in Example 6.

FIG. 9 is a view illustrating a result obtained by taking out a carrier used in Example 6 and carrying out a batch test at a temperature of 5° C. to 20° C.

FIG. 10 is a view illustrating an example of a structure of a device in accordance with an embodiment of the present invention for treating nitrogen-containing water.

FIG. 11 is a view illustrating an example of a structure of a device in accordance with another embodiment of the present invention for treating nitrogen-containing water.

### DESCRIPTION OF EMBODIMENTS

### Embodiment 1: Method for Culturing Low Temperature-Resistant Nitrifying Bacteria

A method in accordance with an embodiment of the present invention for culturing low temperature-resistant nitrifying bacteria is a method including the step of: adjusting a pH of nitrogen-containing water a containing ammonia nitrogen and/or organic nitrogen to not more than 6.0 to obtain nitrogen-containing water b in which a dominant species of microorganisms is low temperature-resistant nitrifying bacteria.

In the present specification, “low temperature-resistant nitrifying bacteria” means bacteria that are capable of nitrifying ammonia nitrogen and/or organic nitrogen contained in nitrogen-containing water in an environment in which a water temperature is not higher than 15° C. A “method for culturing low temperature-resistant nitrifying bacteria” does not mean that only low temperature-resistant nitrifying bacteria are cultured. Rather, bacteria other than low temperature-resistant nitrifying bacteria may be contained in a cultured bacterial colony as long as the low temperature-resistant nitrifying bacteria are successfully cultured. In fact, it is known that it is difficult at present to isolate low temperature-resistant nitrifying bacteria.

The low temperature-resistant nitrifying bacteria are not limited to a specific one but are preferably comammox Nitrospira. Examples of other bacteria encompassed in the low temperature-resistant nitrifying bacteria include Nitrosomonas.

In the present specification, comammox Nitrospira refers to a bacterium which belongs to the genus Nitrospira and which is capable of producing nitric acid directly from ammonia and/or urea contained in nitrogen-containing water. Comammox Nitrospira does not need to produce nitrous acid in order to nitrify ammonia and is thus capable of efficient nitrification.

In the present specification, nitrogen-containing water that contains ammonia nitrogen and/or organic nitrogen and that has not been subjected to the method in accordance with an embodiment of the present invention for culturing low temperature-resistant nitrifying bacteria is referred to as “nitrogen-containing water a containing ammonia nitrogen and/or organic nitrogen”.

Examples of the nitrogen-containing water a include, but are not limited to, domestic wastewater, human waste, factory effluent, and livestock barn-derived wastewater. The nitrogen-containing water a is not necessarily limited to sewage but may be clean water. Ammonia nitrogen (NH₄—N) is a nitrogen compound obtained by decomposition of a nitrogen-containing organic substance such as proteins. Organic nitrogen is converted to ammonia nitrogen by catabolism of BOD oxidative bacteria. It is preferable that the nitrogen-containing water a contains activated sludge.

As a method for culturing low temperature-resistant nitrifying bacteria such as comammox Nitrospira, for example, the method disclosed in Patent Literature 1 is conventionally known. However, this method uses a large amount of a culture medium containing highly-concentrated ammonia. The method therefore cannot be said to be a simple method and involves enormous culturing equipment.

The inventor of the present invention conducted diligent study on a method for culturing low temperature-resistant nitrifying bacteria by a simpler method with use of simple equipment. As a result, the inventor of the present invention made a discovery that a simple method of adjusting a pH of nitrogen-containing water a containing ammonia nitrogen and/or organic nitrogen to not more than 6.0 makes it possible to cause low temperature-resistant nitrifying bacteria to be a dominant species of microorganisms in the nitrogen-containing water.

The method of adjusting the pH of the nitrogen-containing water a to not more than 6.0 can be, for example, a method of aerating the nitrogen-containing water a in a tank. This causes oxygen to be supplied to the nitrogen-containing water a and causes ammonia in the nitrogen-containing water a to change to nitric acid, so that the pH of the nitrogen-containing water a decreases. Thus, the pH is successfully adjusted to not more than 6.0.

Thus, aerating the nitrogen-containing water a allows the pH of the nitrogen-containing water a to be adjusted to not more than 6.0. The low temperature-resistant nitrifying bacteria are aerobic bacteria. The method for culturing low temperature-resistant nitrifying bacteria is capable of creating an aerobic atmosphere by aerating the nitrogen-containing water a and of adjusting the pH of the nitrogen-containing water a to be a value suitable for growth of low temperature-resistant nitrifying bacteria, and can therefore be said to be an efficient method.

However, the method of adjusting the pH of the nitrogen-containing water a is not limited to the method of aerating the nitrogen-containing water a. For example, an acid storage tank and/or an alkali storage tank may be connected to the tank, and the pH of the nitrogen-containing water a may be adjusted by an acid and/or an alkali. Further, also in the case of aerating the nitrogen-containing water a, connecting an acid storage tank and/or an alkali storage tank to the tank allows fine adjustment of the pH to be carried out easily.

The tank is not limited to a specific one. For example, a culture tank for carrying out the above step may be provided, or an existing nitrification tank may be used.

The pH of the nitrogen-containing water a only needs to be not more than 6.0 but is more preferably not more than 5.5. Adjustment of the pH to not more than 5.5 makes it possible to further reduce time until low temperature-resistant nitrifying bacteria become the dominant species in the nitrogen-containing water a and also to more stably cause the low temperature-resistant nitrifying bacteria to become the dominant species. This allows the nitrogen-containing water a to be treated further efficiently.

The pH is preferably more than 3.0. The lower the pH is, the easier it tends to be for low temperature-resistant nitrifying bacteria to be the dominant species in the nitrogen-containing water a. The higher the pH is, the higher the nitrification activity of cultured low temperature-resistant nitrifying bacteria tends to be. As such, the pH is preferably a pH that enables both efficient growth of low temperature-resistant nitrifying bacteria and a high nitrification activity of the low temperature-resistant nitrifying bacteria. From this perspective, the pH is preferably more than 3.0 and more preferably not less than 5.0.

It is preferable that the nitrogen-containing water a contains a carrier. Since carriers are able to retain bacteria, growth and collection of low temperature-resistant nitrifying bacteria are easier in a case where a carrier is contained. The carrier is not limited to a specific one and may be an ordinary carrier such as an adhesion immobilization carrier, an entrapping immobilization carrier, or the like.

As the adhesion immobilization carrier, for example, a carrier in a spherical form, a tubular form, a gelatinous form, or the like, a string-like material, a nonwoven fabric material, and the like may be used. These have many surface irregularities and therefore are preferable in terms of causing bacteria to adhere thereto.

The entrapping immobilization carrier immobilizes bacteria in an entrapping manner such that: bacteria and an immobilization material are mixed; and the immobilization material is polymerized so that the bacteria is entrapped and immobilized inside a resultant gel. Examples of the immobilization material include a monomer material and a prepolymer material. Examples of the monomer material include acrylamide, methylenebisacrylamide, and triacrylformal. Examples of the prepolymer material include polyvinyl alcohol (PVA), polyethyleneglycol diacrylate, polyethyleneglycol methacrylate, and derivatives thereof. Among these, a PVA carrier can be suitably used due to having a mesh structure of not more than 20 μm and being capable of retaining bacteria highly densely.

From the perspective of efficiency in culturing of low temperature-resistant nitrifying bacteria, a proportion of the carrier filling the nitrogen-containing water a is preferably 1% by volume to 60% by volume, more preferably 5% by volume to 20% by volume, and particularly preferably 8% by volume to 15% by volume with respect to a volume of the nitrogen-containing water a in the tank.

Time required until the dominant species of microorganisms contained in the nitrogen-containing water a is low temperature-resistant nitrifying bacteria is approximately 20 days from the day on which the pH of the nitrogen-containing water a becomes not more than 6.0, although the time varies depending on an amount of the nitrogen-containing water a, a specific value of the pH, and the like.

During the adjustment of the pH of the nitrogen-containing water a to not more than 6.0, a temperature of the nitrogen-containing water a is not particularly limited but is preferably a normal temperature (20° C. to 25° C.), from the perspective of improving a growth rate of the low temperature-resistant nitrifying bacteria.

A concentration of the ammonia nitrogen and/or the organic nitrogen in the nitrogen-containing water a is preferably not more than 2000 mg-N/L, more preferably not more than 100 mg-N/L, and particularly preferably not more than 40 mg-N/L.

With the above configuration, a load on the low temperature-resistant nitrifying bacteria during the culturing of the low temperature-resistant nitrifying bacteria is appropriate, and culturing of the low temperature-resistant nitrifying bacteria is carried out efficiently. From this perspective, the concentration is more than 0, preferably not less than 5 mg-N/L, and more preferably not less than 30 mg-N/L.

In the present specification, nitrogen-containing water obtained by the method in accordance with an embodiment of the present invention for culturing low temperature-resistant nitrifying bacteria is referred to as nitrogen-containing water b. The nitrogen-containing water b is nitrogen-containing water which is obtained from the nitrogen-containing water a by causing the dominant species of microorganisms contained in the nitrogen-containing water a to be low temperature-resistant nitrifying bacteria.

In the nitrogen-containing water b, a ratio of an amount of the low temperature-resistant nitrifying bacteria to an amount of ammonia-oxidizing bacteria is preferably more than 1.

In a case where ammonia-oxidizing bacteria are the dominant species in the nitrogen-containing water b, nitrification of ammonia does not progress well at low temperatures. With the above configuration, the low temperature-resistant nitrifying bacteria are a species dominant over the ammonia-oxidizing bacteria in the nitrogen-containing water b. This makes it possible to efficiently carry out nitrification of ammonia at low temperatures. Note that the above ratio can be checked by, for example, analyzing a bacterial colony carried by the carrier by real-time PCR to calculate the ratio.

In the nitrogen-containing water b, the ratio of the amount of the low temperature-resistant nitrifying bacteria to the amount of the ammonia-oxidizing bacteria is more preferably not less than 5 and not more than 100. With the above configuration, the low temperature-resistant nitrifying bacteria are a species significantly dominant over the ammonia-oxidizing bacteria in the nitrogen-containing water b. This makes it possible to even more efficiently carry out nitrification of ammonia at low temperatures.

The nitrogen-containing water b, in which the low temperature-resistant nitrifying bacteria are made the dominant species, can be used suitably for nitrification of ammonia at low temperatures. Therefore, according to the method in accordance with an embodiment of the present invention for culturing low temperature-resistant nitrifying bacteria, it is possible, by a simple method, to: cause low temperature-resistant nitrifying bacteria to be a dominant species; and thus contribute to improvement of efficiency in nitrification at low temperatures.

### Embodiment 2: Method for Treating Nitrogen-Containing Water

A method in accordance with an embodiment of the present invention for treating nitrogen-containing water includes a step of nitrifying, at a water temperature of not lower than 0° C. and not higher than 15° C. in an aerobic atmosphere with use of low temperature-resistant nitrifying bacteria obtained by the method in accordance with an embodiment of the present invention for culturing low temperature-resistant nitrifying bacteria, ammonia contained in the nitrogen-containing water a and/or the nitrogen-containing water b.

Examples of the above step include the following steps.
A step in which (i) nitrogen-containing water b or a carrier which has been filling nitrogen-containing water b is introduced into a nitrification tank which stores therein nitrogen-containing water a, and (ii) nitrification is carried out at a water temperature of not lower than 0° C. and not higher than 15° C. in an aerobic atmosphere.
A step in which (i) the method in accordance with an embodiment of the present invention for culturing low temperature-resistant nitrifying bacteria is carried out in a nitrification tank storing therein nitrogen-containing water a to obtain nitrogen-containing water b, and (ii) nitrification is carried out in the nitrification tank in an aerobic atmosphere with respect to the nitrogen-containing water b having a temperature of not lower than 0° C. and not higher than 15° C.

The aerobic atmosphere can be created, for example, by aerating the nitrogen-containing water a and/or the nitrogen-containing water b.

The “water temperature of not lower than 0° C. and not higher than 15° C.” means that the nitrogen-containing water a and/or the nitrogen-containing water b to be subjected to nitrification has a water temperature of not lower than 0° C. and not higher than 15° C. The low temperature-resistant nitrifying bacteria to be subjected to the method for treating nitrogen-containing water has a high nitrification activity at low temperatures. As such, the water temperature is more preferably not lower than 0° C. and not higher than 10° C. and particularly preferably not lower than 0° C. and not higher than 5° C.

A pH of the nitrogen-containing water a and/or the nitrogen-containing water b to be subjected to nitrification is not particularly limited. The nitrogen-containing water a and/or nitrogen-containing water b may be subjected to nitrification as it is/as they are without adjustment of the pH. Since the nitrification is carried out at a low temperature, no bacteria other than low temperature-resistant nitrifying bacteria can grow. As such, regardless of the pH, the low temperature-resistant nitrifying bacteria are able to carry out nitrification to change ammonia into nitric acid while maintaining the status of being the dominant species.

The water temperature need not be artificially caused to be not lower than 0° C. and not higher than 15° C. In a case where the water temperature is high, a conventionally known nitrification method can simply be used. However, there has been no method that is capable of simply and efficiently carrying out nitrification at a water temperature of not lower than 0° C. and not higher than 15° C.

The method in accordance with an embodiment of the present invention for treating nitrogen-containing water is a method which uses low temperature-resistant nitrifying bacteria contained in the nitrogen-containing water b obtained through the above-described method for culturing low temperature-resistant nitrifying bacteria. As described above, low temperature-resistant nitrifying bacteria are the dominant species in the nitrogen-containing water b. As such, the method for treating nitrogen-containing water, when used in a season when the water temperature is low, makes it possible to carry out nitrification efficiently by avoiding a complication such as heating nitrogen-containing water to be subjected to nitrification.

### Embodiment 3: First Device for Treating Nitrogen-Containing Water

A device in accordance with an embodiment of the present invention for treating nitrogen-containing water is a device for treating nitrogen-containing water, including a culture tank and a nitrification tank, wherein:
the culture tank and the nitrification tank are capable of circulating content between each other;
the culture tank (i) is capable of storing therein (a) nitrogen-containing water a containing ammonia nitrogen and/or organic nitrogen and (b) return sludge obtained from the nitrification tank, and (ii) produces nitrogen-containing water b from the nitrogen-containing water a by adjusting a pH of the nitrogen-containing water a to not more than 6.0 and thereby causing a dominant species of microorganisms contained in the nitrogen-containing water a to be low temperature-resistant nitrifying bacteria; and
the nitrification tank (i) is capable of storing therein the nitrogen-containing water a and the nitrogen-containing water b and (ii) produces a nitrification liquid by nitrifying, at a water temperature of not lower than 0° C. and not higher than 15° C. with use of the low temperature-resistant nitrifying bacteria contained in the nitrogen-containing water b, ammonia contained in the nitrogen-containing water a and/or the nitrogen-containing water b.

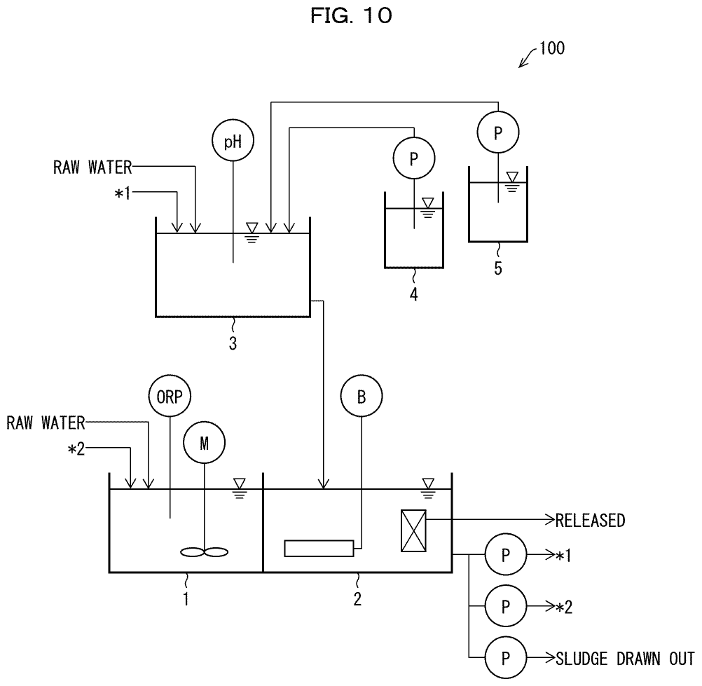

The device for treating nitrogen-containing water is capable of carrying out the method in accordance with an embodiment of the present invention for culturing low temperature-resistant nitrifying bacteria and the method in accordance with an embodiment of the present invention for treating nitrogen-containing water. FIG. 10 is a view illustrating an example of a structure of the device in accordance with the present embodiment for treating nitrogen-containing water.

In FIG. 10, 100 represents a device for treating nitrogen-containing water, 1 represents a denitrification tank, 2 represents a nitrification tank, 3 represents a culture tank, 4 represents an acid storage tank, 5 represents an alkali storage tank, “pH” represents a pH meter, “P” represents a pump, “ORP” represents an oxidation-reduction potentiometer, “B” represents an aeration device, “M” represents a stirring motor, *1 represents introduction of return sludge from the nitrification tank 2 into the culture tank 3, and *2 represents introduction of a nitrification liquid from the nitrification tank 2 into the denitrification tank 1.

“RAW WATER” represents introduction of nitrogen-containing water a into the denitrification tank 1 and the culture tank 3, and an arrow from the culture tank 3 to the nitrification tank 2 represents introduction of nitrogen-containing water b produced in the culture tank 3 into the nitrification tank 2. These introductions may be carried out via introduction sections respectively connected to the denitrification tank 1, the nitrification tank 2, and the culture tank 3. Examples of the introduction section include a pipe.

A material and a volume of each of the denitrification tank 1, the nitrification tank 2, and the culture tank 3 are not particularly limited. Note that although the denitrification tank 1 is illustrated in FIG. 10, the denitrification tank 1 is an optional configuration in the treatment device 100.

The culture tank 3 is capable of carrying out the method described in Embodiment 1, and produces nitrogen-containing water b from the introduced nitrogen-containing water a by adjusting a pH of the nitrogen-containing water a to not more than 6.0 and thereby causing a dominant species of microorganisms contained in the nitrogen-containing water a to be low temperature-resistant nitrifying bacteria. The method of adjusting the pH is as described in Embodiment 1.

The culture tank 3 introduces the nitrogen-containing water b into the nitrification tank 2. In a case where a carrier on which the low temperature-resistant nitrifying bacteria are preferentially caused to grow is present in the nitrogen-containing water b, the carrier may be taken out of the culture tank 3 and introduced alone into the nitrification tank 2. In the nitrification tank 2, a nitrification liquid is produced by nitrifying, at a water temperature of not lower than 0° C. and not higher than 15° C. in an aerobic atmosphere with use of the low temperature-resistant nitrifying bacteria contained in the nitrogen-containing water b, ammonia contained in the nitrogen-containing water a and/or the nitrogen-containing water b. The nitrification is as described in Embodiment 2.

The denitrification tank 1 is capable of storing therein nitrogen-containing water a and the nitrification liquid produced in the nitrification tank 2, and denitrifies nitric acid contained in the nitrification liquid in an anaerobic atmosphere to produce nitrogen gas.

The culture tank 3 and the nitrification tank 2 are configured such that: the culture tank 3 introduces the nitrogen-containing water b and/or the carrier contained in the nitrogen-containing water b into the nitrification tank 2, and the nitrification tank 2 introduces return sludge obtained in the nitrification tank 2 into the culture tank 3.

That is, the culture tank 3 and the nitrification tank 2 are capable of circulating content between each other. The return sludge is acidic and therefore can be used for adjusting a pH of nitrogen-containing water a introduced into the culture tank 3 to 6.0. Further, since the return sludge contains low temperature-resistant nitrifying bacteria, the return sludge may be: returned to the culture tank 3 once; then re-introduced into the nitrification tank 2 together with nitrogen-containing water b newly produced in the culture tank 3; and subjected to nitrification. Since low temperature-resistant nitrifying bacteria are the dominant species in the nitrogen-containing water b, nitrification can progress efficiently in the nitrification tank 2 in an environment with a water temperature of not lower than 0° C. and not higher than 15° C. Thus, in the treatment device 100, by circulating content between the culture tank 3 and the nitrification tank 2, it is possible to continuously carry out culturing of low temperature-resistant nitrifying bacteria and nitrification at a low temperature.

### Embodiment 4: Second Device for Treating Nitrogen-Containing Water

A device in accordance with an embodiment of the present invention for treating nitrogen-containing water is a device for treating nitrogen-containing water, the device including a treatment system A and a treatment system B, wherein:
the treatment system A includes a nitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the nitrification liquid obtained in the nitrification tank I, and a nitrification liquid obtained in the nitrification tank II, (ii) denitrifies nitric acid contained in each of the nitrification liquid obtained in the nitrification tank I and the nitrification liquid obtained in the nitrification tank II, and (iii) transfers the nitrogen-containing water a to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. in an aerobic atmosphere with use of the low temperature-resistant nitrifying bacteria, and (iii) transfers the nitrification liquid to the denitrification tank II.

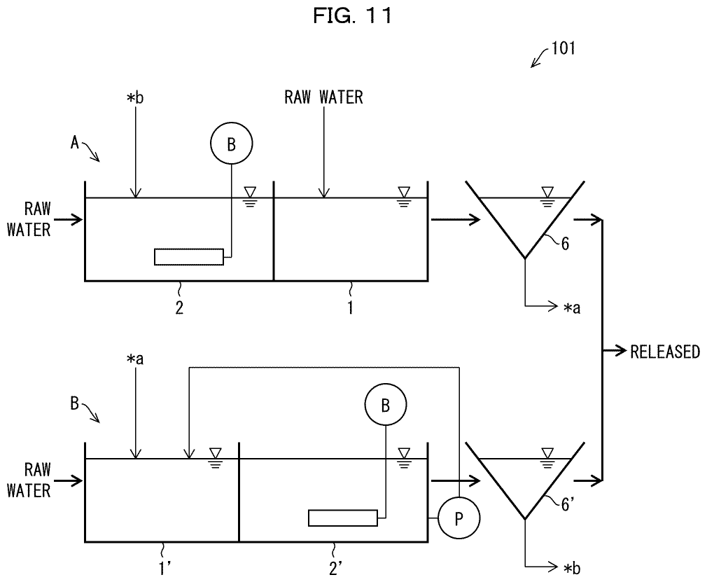

The above treatment device is capable of carrying out the method in accordance with an embodiment of the present invention for culturing low temperature-resistant nitrifying bacteria and the method in accordance with an embodiment of the present invention for treating nitrogen-containing water. FIG. 11 is a view illustrating an example of a structure of the treatment device in accordance with the present embodiment. No further description will be provided as to reference numerals already described.

In FIG. 11, 101 represents a device for treating nitrogen-containing water, A represents a treatment system A, B represents a treatment system B, 1 and 1′ each represent a denitrification tank, 2 and 2′ each represent a nitrification tank, 6 and 6′ each represent a sedimentation tank, *a represents introduction of a nitrification liquid from the sedimentation tank 6 into the denitrification tank 1′, and *b represents introduction of return sludge from the sedimentation tank 6′ into the nitrification tank 2. “RAW WATER” represents introduction of nitrogen-containing water a into the denitrification tank 1, the nitrification tank 2, or the denitrification tank 1′.

Although FIG. 11 illustrates a denitrification tank 1, the treatment device in accordance with the present embodiment does not include the denitrification tank 1. As such, although a treatment liquid is introduced from the denitrification tank 1 into the sedimentation tank 6 in FIG. 11, a nitrification liquid is introduced from the nitrification tank 2 into the sedimentation tank 6 in the present embodiment. Note that an aspect in which the denitrification tank 1 is provided will be described in Embodiment 5.

In the treatment system A, similarly as the culture tank 3, the nitrification tank 2 (corresponding to the nitrification tank I) adjusts a pH of introduced nitrogen-containing water a to not more than 6.0 to cause a dominant species of microorganisms contained in the introduced nitrogen-containing water a to be low temperature-resistant nitrifying bacteria. As a result, nitrogen-containing water b is obtained in the nitrification tank 2. Then, in the nitrification tank 2, nitrification of ammonia contained in the nitrogen-containing water b is carried out at a temperature of not lower than 0° C. and not higher than 15° C. in an aerobic atmosphere, and a nitrification liquid obtained is introduced into the denitrification tank 1′ (corresponding to the denitrification tank II) of the treatment system B via the sedimentation tank 6.

In the treatment system B, the denitrification tank 1′ and the nitrification tank 2′ (corresponding to the nitrification tank II) circulate content between each other. That is, circulation of a nitrification liquid is carried out between the denitrification tank 1′ and the nitrification tank 2′.

Into the denitrification tank 1′, a nitrification liquid (“*a” in FIG. 11) produced in the nitrification tank 2 and a nitrification liquid (an arrow from the nitrification tank 2′ to the denitrification tank 1′) produced in the nitrification tank 2′ are introduced. The denitrification tank 1′ denitrifies nitric acid contained in each of these nitrification liquids in an anaerobic atmosphere to produce nitrogen gas. Further, the denitrification tank 1′ transfers, to the nitrification tank 2′, the nitrogen-containing water a (“RAW WATER” in FIG. 11) introduced into the denitrification tank 1′.

The nitrification tank 2′, similarly as the culture tank 3, adjusts a pH of introduced nitrogen-containing water a to not more than 6.0 to cause a dominant species of microorganisms contained in the introduced nitrogen-containing water a to be low temperature-resistant nitrifying bacteria. As a result, nitrogen-containing water b is obtained in the nitrification tank 2′. Then, in the nitrification tank 2′, nitrification of ammonia contained in the nitrogen-containing water b is carried out at a temperature of not lower than 0° C. and not higher than 15° C. in an aerobic atmosphere, and a nitrification liquid obtained is introduced into the denitrification tank 1′ as described above. A treatment liquid which has been subjected to the denitrification in the denitrification tank 1′ is introduced into the sedimentation tank 6′. Further, return sludge 6′ is introduced into the nitrification tank 2 (*b in FIG. 11).

As described above, in the treatment device 101, the nitrification tank 2 and the nitrification tank 2′ each serve as the culture tank 3 and also carry out nitrification with use of low temperature-resistant nitrifying bacteria which have become a dominant species. Further, the return sludge obtained in the treatment system B contains low temperature-resistant nitrifying bacteria, and introducing the return sludge into the nitrification tank 2 allows the low temperature-resistant nitrifying bacteria to be reused in the nitrification tank 2. Further, the nitrification liquid obtained in the treatment system A is introduced into the denitrification tank 1′ and subjected to denitrification at once together with the nitrification liquid introduced from the nitrification tank 2′.

Thus, the treatment device 101 is capable of continuously carrying out culturing of low temperature-resistant nitrifying bacteria and nitrification at a low temperature, by causing a product obtained in the treatment system A and a product obtained in the treatment system B to circulate.

### Embodiment 5: Third Device for Treating Nitrogen-Containing Water

A device in accordance with an embodiment of the present invention for treating nitrogen-containing water is a device for treating nitrogen-containing water, the device including a treatment system A and a treatment system B, wherein:
the treatment system A includes a nitrification tank I and a denitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the denitrification tank I denitrifies nitric acid contained in the nitrification liquid obtained in the nitrification tank I and transfers obtained return sludge to the treatment system B;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the return sludge from the denitrification tank I, and a nitrification liquid obtained in the nitrification tank II, (ii) denitrifies nitric acid contained in the nitrification liquid, and (iii) transfers the nitrogen-containing water a and the return sludge to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge, and (iii) transfers the nitrification liquid to the denitrification tank II.

An example of a configuration of the treatment device in accordance with the present embodiment is illustrated in FIG. 11. The treatment device differs from the second device for treating nitrogen-containing water by including a denitrification tank 1. As such, whereas a nitrification liquid obtained in the nitrification tank 2 is introduced into the denitrification tank 1′ of the treatment system B in the second device for treating nitrogen-containing water, return sludge obtained in the denitrification tank 1 is introduced into the denitrification tank 1′ in the present embodiment. In the present embodiment, low temperature-resistant nitrifying bacteria contained in the return sludge obtained in the denitrification tank 1 are reused in the nitrification tank 2′, unlike in the second device for treating nitrogen-containing water. The other points are similar to those in the second device for treating nitrogen-containing water, and as in the second treatment device, culturing of low temperature-resistant nitrifying bacteria and nitrification at a low temperature can be continuously carried out by causing a product obtained in the treatment system A and a product obtained in the treatment system B to circulate.

Aspects of the present invention can also be expressed as follows:

The present invention includes the following aspects.
<1> A method for culturing low temperature-resistant nitrifying bacteria, the method including the step of: adjusting a pH of nitrogen-containing water a containing ammonia nitrogen and/or organic nitrogen to not more than 6.0 to obtain nitrogen-containing water b in which a dominant species of microorganisms is low temperature-resistant nitrifying bacteria.
<2> The method described in <1>, wherein the pH of the nitrogen-containing water a is adjusted to more than 3.0 and not more than 6.0.
<3> The method described in <1> or <2>, wherein a concentration of the ammonia nitrogen and/or the organic nitrogen in the nitrogen-containing water a is not more than 2000 mg-N/L.
<4> The method described in any one of <1> to <3>, wherein a ratio of an amount of the low temperature-resistant nitrifying bacteria to an amount of ammonia-oxidizing bacteria is more than 1 in the nitrogen-containing water b.
<5> The method described in <4>, wherein the ratio is not less than 5 and not more than 100.
<6> The method described in any one of <1> to <5>, wherein the low temperature-resistant nitrifying bacteria are comammox Nitrospira.
<7> A method for treating nitrogen-containing water, the method including the step of: nitrifying, at a water temperature of not lower than 0° C. and not higher than 15° C. in an aerobic atmosphere with use of low temperature-resistant nitrifying bacteria obtained by a method described in any one of <1> to <6>, ammonia contained in the nitrogen-containing water a and/or the nitrogen-containing water b.
<8> A device for treating nitrogen-containing water, the device including a culture tank and a nitrification tank, wherein:
   the culture tank and the nitrification tank are capable of circulating content between each other;
   the culture tank (i) is capable of storing therein (a) nitrogen-containing water a containing ammonia nitrogen and/or organic nitrogen and (b) return sludge obtained from the nitrification tank, and (ii) produces nitrogen-containing water b from the nitrogen-containing water a by adjusting a pH of the nitrogen-containing water a to not more than 6.0 and thereby causing a dominant species of microorganisms contained in the nitrogen-containing water a to be low temperature-resistant nitrifying bacteria; and
   the nitrification tank (i) is capable of storing therein the nitrogen-containing water a and the nitrogen-containing water b and (ii) produces a nitrification liquid by nitrifying, at a water temperature of not lower than 0° C. and not higher than 15° C. with use of the low temperature-resistant nitrifying bacteria contained in the nitrogen-containing water b, ammonia contained in the nitrogen-containing water a and/or the nitrogen-containing water b.
<9> A device for treating nitrogen-containing water, the device including a treatment system A and a treatment system B, wherein:
   the treatment system A includes a nitrification tank I;
   the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
   the treatment system B includes a nitrification tank II and a denitrification tank II;
   the nitrification tank II and the denitrification tank II are capable of circulating content between each other;
   the denitrification tank II (i) is capable of storing therein nitrogen-containing water a containing ammonia nitrogen and/or organic nitrogen, the nitrification liquid obtained in the nitrification tank I, and a nitrification liquid obtained in the nitrification tank II, (ii) denitrifies nitric acid contained in each of the nitrification liquid obtained in the nitrification tank I and the nitrification liquid obtained in the nitrification tank II, and (iii) transfers the nitrogen-containing water a to the nitrification tank II; and
   the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. in an aerobic atmosphere with use of the low temperature-resistant nitrifying bacteria, and (iii) transfers the nitrification liquid to the denitrification tank II.
<10> A device for treating nitrogen-containing water, the device including a treatment system A and a treatment system B, wherein:
   the treatment system A includes a nitrification tank I and a denitrification tank I;
   the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
   the denitrification tank I denitrifies nitric acid contained in the nitrification liquid obtained in the nitrification tank I and transfers obtained return sludge to the treatment system B;
   the treatment system B includes a nitrification tank II and a denitrification tank II;
   the nitrification tank II and the denitrification tank II are capable of circulating content between each other;
   the denitrification tank II (i) is capable of storing therein nitrogen-containing water a containing ammonia nitrogen and/or organic nitrogen, the return sludge from the denitrification tank I, and a nitrification liquid obtained in the nitrification tank II, (ii) denitrifies nitric acid contained in the nitrification liquid, and (iii) transfers the nitrogen-containing water a and the return sludge to the nitrification tank II; and
   the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge, and (iii) transfers the nitrification liquid to the denitrification tank II.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

The following will describe examples of the present invention.

### Example 1

In Example 1, study was conducted as to whether or not setting a pH of nitrogen-containing water containing ammonia nitrogen and organic nitrogen to 5 would make it possible to cause a dominant species of microorganisms contained in the nitrogen-containing water to be low temperature-resistant nitrifying bacteria.

As the nitrogen-containing water containing nitrifying bacteria, sewage from a sewage treatment plant (located in Gunma Prefecture), at which a general activated sludge process is practiced, was used. A concentration of the ammonia nitrogen contained in the nitrogen-containing water to be treated was approximately 48 mg/L.

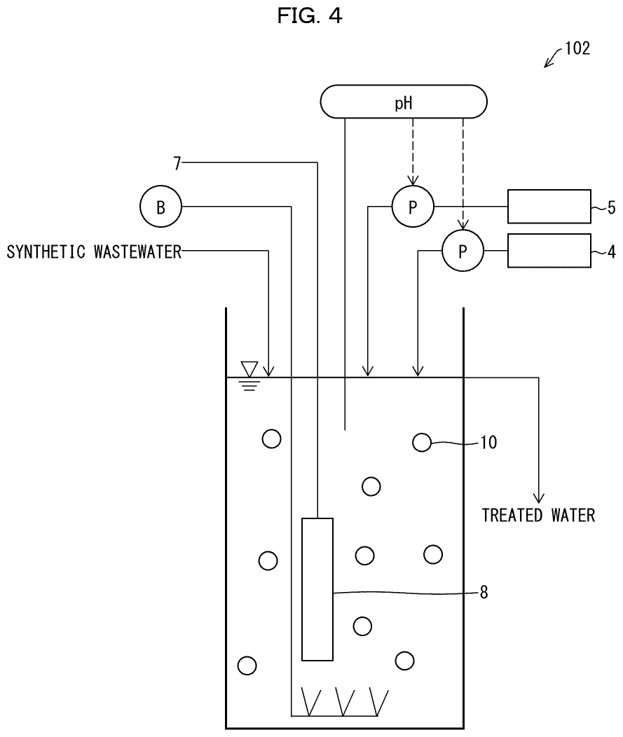

Into a continuously operating device 102 illustrated in FIG. 4, return sludge from the sewage treatment plant was introduced in an amount equal to 90% of a volume of a water tank, and a carrier 10 was introduced in an amount equal to 10% of the volume of the water tank. The nitrogen-containing water to be treated was caused to pass through at a flow rate with which 100% of the volume of the water tank was replaced in a day. The water tank of the continuously operating device 102 had an effective volume of 1.1 L. In FIG. 4, 4 represents an acid storage tank, 5 represents an alkali storage tank, 7 represents a thermocouple, 8 represents a heater, 10 represents a carrier, “B” represents an aeration device, “P” represents a pump, and “PH” represents a pH controller. In the continuously operating device 102, air was blown into the nitrogen-containing water from the aeration device to lower the pH of the nitrogen-containing water to 5. The acid storage tank 4 had 0.5 N hydrochloric acid stored therein, and the alkali storage tank had 5% (W/V) NaHCO₃ stored therein. The pH controller controlled the acid storage tank and the alkali storage tank so as to introduce the hydrochloric acid or the NaHCO₃ into the tank as necessary to maintain the pH of the nitrogen-containing water at 5, and the water temperature was maintained at 20° C. to 25° C.

The carrier 10 used was a polyvinyl alcohol (PVA) sponge carrier (manufactured by AION Co., Ltd., 4 mm square).

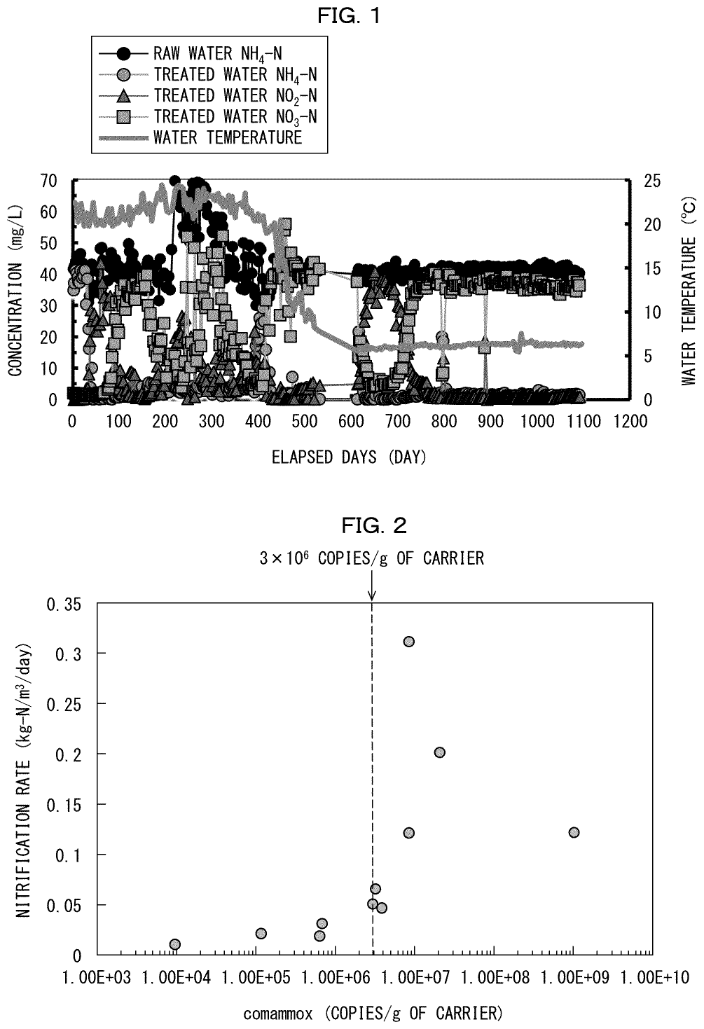

Subsequently, as illustrated in FIG. 1, the water temperature was reduced to 5° C. on Day 448 of operation. Then, the water temperature was maintained at approximately 5° C. to 6° C. FIG. 1 illustrates a result of nitrifying, in an aerobic atmosphere, ammonia contained in the nitrogen-containing water. In FIG. 1, “RAW WATER NH₄—N” represents ammonia nitrogen contained in the nitrogen-containing water, “TREATED WATER NH₄—N” represents ammonia nitrogen in treated water (nitrification liquid), “TREATED WATER NO₂—N” represents nitrite-nitrogen in the treated water, and “TREATED WATER NO₃—N” represents nitrate-nitrogen in the treated water.

Concentrations of “RAW WATER NH₄—N”, “TREATED WATER NH₄—N”, “TREATED WATER NO₂—N”, and “TREATED WATER NO₃—N” were measured in accordance with Gesui Shiken Hoho (Sewage Test Methods) (Japan Sewage Works Association, 2012).

As illustrated in FIG. 1, in a state where the water temperature was maintained at 5° C. to 6° C., the concentration of NO₃—N (nitrogen derived from nitric acid) in the treated water (nitrification liquid) increased from around Day 650 and reached a plateau around Day 770, and NO₃—N was stably obtained thereafter until Day 1100. This result suggests that setting the pH of the nitrogen-containing water to 5 caused low temperature-resistant nitrifying bacteria to be a dominant species in the nitrogen-containing water, and that stable nitrification was therefore achieved even though the water temperature decreased to 5° C. to 6° C. later.

A nitrification rate at 6° C. was 0.12 kg-N/m³/day. The nitrification rate was determined in accordance with the above Sewage Test Method.

Further, a bacterial colony of the carrier after the low temperature-resistant nitrifying bacteria had become the dominant species in the nitrogen-containing water was analyzed by real-time PCR. As primers, those indicated in Table 1 were used.

DNA extraction from the carrier was carried out with use of DNeasy PowerBiofilm Kit (Qiagen). In the real-time PCR, environmental samples were used, and products obtained by purifying respective target genes were used as standards. TB Green (registered trademark) Premix Ex Taq (trademark) II (Tli RNaseH Plus) (Takara-Bio Inc.) was used for the measurement, and StepOnePlus (trademark) Real-time PCR (Applied Biosystems) was used as a real-time PCR device.

In Table 1, Beta-AOB represents general ammonia-oxidizing bacteria, AOA represents ammonia-oxidizing archaea, comammox represents comammox Nitrospira, and Bacteria represents the entire bacteria.

TABLE 1

| | | | | SEQ | Concen- | Annealing | |
|---|---|---|---|---|---|---|---|
| | | Primer/ | Sequence | ID | tration of | temperature | |
| Target | Gene | probe | (5' to 3') | NO | primer (μM) | (° C.) | Reference |
| Beta-AOB | amoA | amoA-1F | GGGGTTTCTACTGGTGGT | 1 | 0.3 | 58.5 | Rotthauwe et |
| | | | | | | | al., |
| | | | | | | | 1997 1) |
| | | amoA-rNew | CCCCTCBGSAAAVCCTTC | 2 | | | Hornek et |
| | | | TTC | | | | al., 2006 2) |
| AOA | amoA | Arch-amoA- | CTGAYTGGGCYTGGACATC | 2 | 0.15 | 58.5 | Wuchter et. |
| | | for | | | | | al. |
| | | Arch-amoA- | TTCTTCTTTGTTGCCCAGT | 4 | | | 2006 3) |
| | | rev | A | | | | |
| comammox | amoA | Ntsp-amoA | GGATTTCTGGNTSGATTGG | 5 | 0.15 | 48 | Fowler et |
| | | 162F | A | | | | al., |
| | | Ntsp-amoA | WAGTTNGACCACCASTACC | 6 | | | 2018 4) |
| | | 359R | A | | | | |
| Bacteria | 16S | BACT1369F | CGGTGAATACGTTCYCGG | 7 | 0.15 | 56.5 | Suzuki et |
| | rRNA | PROK1492R | CGWTACCTTGTTACGACTT | 8 | | | al., |
| | | Tm1389F | CTTGTACACACCGCCCGTC | 9 | | | 2000 5) |

1) Appl. Environ. Microbiol., 63, 4704-4712 (1997)
2) J Microbiol Methods. 2006 July; 66(1): 147-55
3) Proc. Natl. Acad. Sci. U.S.A. 103, 12317-12322 (2006)
4) Environ Microbiol. 2018 March; 20(3): 1002-1015.
5) Appl Environ Microbiol. 2000 November; 66(11): 4605-14

As a result of analysis by real-time PCR, comammox Nitrospira was detected in a greater amount than the ammonia-oxidizing bacteria. It is therefore considered that in the present example, the adjustment of the pH of the nitrogen-containing water to 5 caused comammox Nitrospira to be the dominant species in the nitrogen-containing water. It is also considered that because comammox Nitrospira had become the dominant species, stable nitrification was achieved even at the low temperature of 5° C. to 6° C.

In a case of carrying out nitrification in an ordinary wastewater treatment, a pH of nitrogen-containing water is maintained high in order to prevent unsatisfactory nitrification caused by accumulation of nitrous acid and inhibition of nitrification by the nitrous acid. The present study has made a new discovery that adjustment of the pH of nitrogen-containing water to not more than 6.0 inhibits the usual nitrification with nitrous acid production and makes it possible to cause low temperature-resistant nitrifying bacteria to be a dominant species of microorganism in the nitrogen-containing water. The low temperature-resistant nitrifying bacteria are capable of nitrification even in a low-temperature environment and are capable of causing ammonia to be oxidized into nitric acid without going through a nitrous acid phase. The result of the present example indicates that according to the method in accordance with an embodiment of the present invention for culturing low temperature-resistant nitrifying bacteria, it is possible, by a simple method, to: cause low temperature-resistant nitrifying bacteria to be a dominant species; and thus carry out nitrification while inhibiting production of nitrous acid in a low-temperature environment.

### Example 2

In the present example, study was conducted on an amount of low temperature-resistant nitrifying bacteria necessary for carrying out nitrification at 5° C.

To a nitrification tank having an MLSS of 2000 mg/L (suspended sludge), a carrier was added in an amount of 10% by volume with respect to a volume of the nitrification tank. Comammox Nitrospira successfully cultured was carried in an amount of 1×10⁴ copies/g to 1×10⁹ copies/g of the carrier. Amounts of bacteria carried are indicated along a horizontal axis in FIG. 2. Sewage used had a composition indicated in Table 4. The carrier used was a PVA sponge carrier as in Example 1.

FIG. 2 shows the results. With a carrier introduction ratio of 0.1 L/L of the water tank, a low nitrification rate was exhibited in cases where the amount of comammox Nitrospira carried was 1×10⁴ copies/g to 1×10⁶ copies/g, but a nitrification rate of 0.05 kg-N/m³/day was exhibited in a case where the amount of comammox Nitrospira carried was 3×10⁶ copies/g. Further, a nitrification rate of more than 0.1 kg-N/m³/day was exhibited for 8×10⁶ copies/g, and then nitrification rates of more than 0.1 kg-N/m³/day were exhibited for up to 1×10⁹ copies/g.

Thus, it was found out that in a case of carrying out nitrification by a method in which comammox Nitrospira was carried by a carrier (carrier method), a bacterial count of 3×10⁶ copies/g was necessary in order to satisfy a nitrification rate of 0.05 kg-N/m³/day.

Note here that the carrier carrying comammox Nitrospira in an amount of 3×10⁶ copies per gram of the carrier was added in an amount of 10% by volume with respect to the volume of the nitrification tank. It thus follows that in a case of carrying out nitrification by an activated sludge process, it is necessary to use comammox Nitrospira in an amount of 3×10⁵ copies per milliliter of the volume of the nitrification tank in order to achieve a performance equivalent to 0.05 kg-N/m³/day.

### Example 3

In the present example, sewage was treated by a modified activated sludge process to confirm whether or not using comammox Nitrospira in a floatation method was advantageous. The modified activated sludge process is a method which uses a nitrification tank and a denitrification tank and in which a nitrification liquid is circulated between the nitrification tank and the denitrification tank. Operation conditions are indicated in Table 2.

TABLE 2

| | |
|---|---|
| Sewage | BOD: 120 mg/L, NH₄—N: 30 mg/L-38 mg/L |
| Sewage residence | 6 h |
| time | |
| Water temperature | 10° C. |
| Nitrification liquid | 3 times (relative to amount of sewage |
| return ratio | treated) |
| Sludge return ratio | 1 time (relative to amount of sewage |
| | treated) |
| Amount of | Culture fluid with 8 × 10⁹ copies/mL was |
| comammox | added to nitrification tank in an amount of |
| bacteria added | 1/4000 by volume (relative to amount of |
| | sewage treated) |

A T-N removal ratio of not less than 75% was exhibited. Further, as a conventional method, an operation was carried out under the same conditions as in Example 3 except for not adding comammox Nitrospira. In this case, the T-N removal ratio was not more than 40%.

### Example 4

In the present example, study was conducted on growth properties of comammox Nitrospira, with use of a carrier on which comammox Nitrospira was dominant. That is, study was conducted as to conditions for increasing a seed carrier.

As a device, the continuously operating device 102 illustrated in FIG. 4 was used. Operation conditions are indicated in Table 3.

TABLE 3

| | |
|---|---|
| Inorganic | NH₄—N: 40 mg/L |
| synthetic | |
| wastewater | |
| Residence | 12 h (Run 1) −> 8 h (Run 2) −> 6 h (Run 3) −> 4 h |
| time of | (Run 4). |
| inorganic | Each run lasted for 2 weeks, total of 8 weeks |
| synthetic | |
| wastewater | |
| Water | 25° C. |
| temperature | |
| Seed | Obtained by causing comammox Nitrospira to be |
| bacteria | carried by PVA sponge carrier (manufactured by |
| (seed- | AION Co., Ltd., 5-mm-square carrier) as seed |
| carrying | bacteria in an amount of 1.8 × 10⁹ copies/g. Used |
| carrier) | in an amount of 1% by volume relative to volume |
| | of suspended sludge in inorganic synthetic |
| | wastewater. |
| Seed- | Brand-new PVA sponge carrier (manufactured by |
| uncarrying | AION Co., Ltd., 5-mm-square carrier) was used in |
| carrier | an amount of 9% by volume relative to volume of |
| | suspended sludge in inorganic synthetic |
| | wastewater |

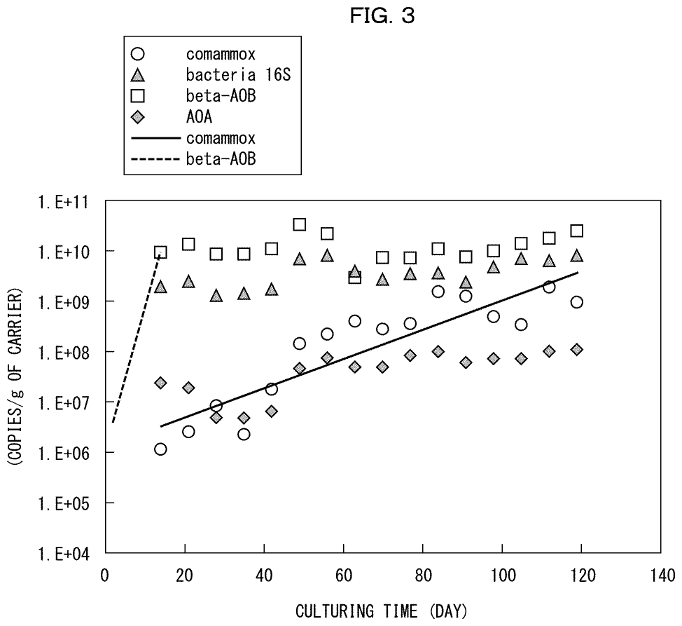

A carrier serving as seed bacteria (Enobi et al. (2021), Japanese Journal of Water Treatment Biology, 57, 35-41) and a brand-new sponge carrier which are indicated in Table 3 were introduced into the continuously operating device 102, and an operation was carried out from Run 1 through Run 4 for a total of 8 weeks, in which each run was carried out for 2 weeks. On the final day of each run, the PVA sponge carriers were collected and subjected to analysis of a bacterial colony with use of real-time PCR by a method similar to that in Example 1. Results are shown in FIG. 3.

FIG. 3 is a view illustrating a result of studying growth properties of comammox Nitrospira at 25° C. with use of the carrier serving as seed bacteria. In FIG. 3, comammox represents a bacteria count of comammox Nitrospira, bacteria 16S represents a bacterial count of the entire eubacteria, beta-AOB represents a bacterial count of ammonia-oxidizing bacteria, and AOA represents a bacterial count of ammonia-oxidizing archaea.

Comammox Nitrospira had a specific growth rate u of 0.124/day and a doubling time of 5.58 days. The specific growth rate of comammox Nitrospira was 1/30 of that of the ammonia-oxidizing bacteria. It was thus found that the ammonia-oxidizing bacteria became the dominant species under the water temperature condition of 25° C. However, since the doubling time of comammox Nitrospira was 5.58 days, it is possible, even under the above conditions, to cause comammox Nitrospira to grow by operating with a sludge residence time (SRT) of not less than 5.58 days.

### Example 5

In the present example, study was conducted on appropriate conditions for culturing low temperature-resistant nitrifying bacteria. As a device, the continuously operating device 102 illustrated in FIG. 4 was used. Inorganic synthetic wastewater used had a composition indicated in Table 4, and operation conditions employed are indicated in Table 5.

TABLE 4

| | Composition |
|---|---|
| Chemical name | (mg/L) |
| NH₄Cl | 150 |
| NaHCO₃ | 244 |
| Na₂HPO₄•12H₂O | 46 |

TABLE 5

| | |
|---|---|
| Inorganic | Composition: Table 4, NH₄—N: 40 mg/L, pH: 6 |
| synthetic | |
| wastewater | |
| Residence | 6 h-24 h |
| time of | |
| inorganic | |
| synthetic | |
| wastewater | |
| Water | 25° C. |
| temperature | |
| Seed | Obtained by causing comammox Nitrospira to be |
| bacteria | carried by PVA sponge carrier (manufactured by |
| (carrier | AION Co., Ltd., 5-mm-square carrier) as seed |
| carrying | bacteria in an amount of 1.8 × 10⁹ copies/g. Used |
| seed) | in an amount of 1% by volume relative to volume |
| | of suspended sludge in inorganic synthetic |
| | wastewater. |
| carrier | Brand-new PVA sponge carrier (manufactured by |
| uncarrying | AION Co., Ltd., 5-mm-square carrier) was used in |
| seed | an amount of 9% by volume relative to volume of |
| | suspended sludge in inorganic synthetic |
| | wastewater |
| Aeration | 1 L/min |
| amount | |

A carrier serving as seed bacteria (Enobi et al. (2021), Japanese Journal of Water Treatment Biology, 57, 35-41) and a brand-new sponge carrier which are indicated in Table 5 were introduced into the continuously operating device 102 containing the inorganic synthetic wastewater indicated in Table 4. A pH of the inorganic synthetic wastewater was adjusted to 6 by blowing air from an aeration device into the inorganic synthetic wastewater. The time when the pH of the inorganic synthetic wastewater became 6 was considered Day 0 of operation, and the pH was maintained at 6 until Day 149 to allow for acclimatization.

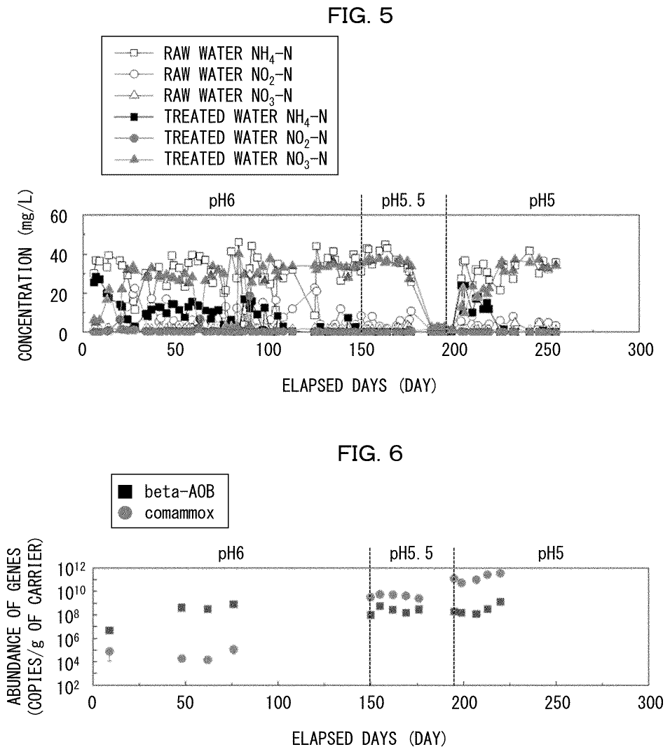

FIG. 5 is a view illustrating a result of checking a change over time in quality of the inorganic synthetic wastewater in an acidic environment. In FIG. 5, “RAW WATER NH₄—N” represents ammonia nitrogen contained in the inorganic synthetic wastewater, “RAW WATER NO₂—N” represents nitrite-nitrogen contained in the inorganic synthetic wastewater, “RAW WATER NO₃—N” represents nitrate-nitrogen contained in the inorganic synthetic wastewater, “TREATED WATER NH₄—N” represents ammonia nitrogen in treated water (nitrification liquid), “TREATED WATER NO₂—N” represents nitrite-nitrogen in the treated water, and “TREATED WATER NO₃—N” represents nitrate-nitrogen in the treated water.

Concentrations of “RAW WATER NH₄—N” etc. were measured by a method in accordance with Gesui Shiken Hoho (Sewage Test Methods) (Japan Sewage Works Association, 2012).

Then, the pH of the inorganic synthetic wastewater was changed to 5.5 and further to 5 after an elapse of respective days indicated in FIG. 5, and approximately 60 days of operation was carried out for each case. During the period of operation, a water temperature was maintained at 25° C. and dissolved oxygen was maintained at 7 mg/L to 8 mg/L, and the water quality was analyzed twice a week.

As indicated in FIG. 5, in an initial stage of acclimatization (up to Day 100), removal of ammonia was observed as well as production of nitric acid, but a nitrification ratio was approximately 54.19% (the nitrification ratio is not indicated in FIG. 5). From Day 100 to Day 149 (pH 6.0), the water quality was stable, and the nitrification ratio reached 95.44%. At pH 5.5 (Day 150 to Day 176), the nitrification ratio was 100%, and as described later, a standing crop of comammox Nitrospira surpassed that of the ammonia-oxidizing bacteria. During an operation at pH 5.0 (Day 177 and after), a slight generation of nitrous acid was observed immediately after the condition was changed, but a more stable water quality (an average nitrification ratio of 98.66%) was achieved after a long time of operation (Day 225 and after).

FIG. 6 is a view illustrating a result of real-time PCR of a bacterial colony carried by a carrier in the inorganic synthetic wastewater having a pH adjusted to 6, 5.5, or 5. The horizontal axis represents days counted from a start of the operation, and the vertical axis represents an abundance of genes per gram of the carrier. Further, beta-AOB represents general ammonia-oxidizing bacteria, and comammox represents comammox Nitrospira.

Comammox Nitrospira and the ammonia-oxidizing bacteria in the carrier were subjected to quantification of amoA genes by real-time PCR with use of specific primers. From a carrier collected on a day indicated along the horizontal axis in FIG. 6, DNA extraction was carried out with use of DNeasy PowerBiofilm Kit (Qiagen). In the real-time PCR, an environmental sample was used, and products obtained by purifying respective target genes were used as standards. TB Green (registered trademark) Premix Ex Taq (trademark) II (Tli RNaseH Plus) (Takara-Bio Inc.) was used for the measurement, and StepOnePlus (trademark) Real-time PCR (Applied Biosystems) was used as a real-time PCR device. Note that the specific primers were the same as those indicated for “Beta-AOB” and “comammox” in Table 1.

As illustrated in FIG. 6, comammox Nitrospira accumulated only weakly from a start to around Day 80 of operation. However, from Day 150, on which the pH was adjusted to 5.5, it was confirmed that comammox Nitrospira was the dominant species. The results shown in FIG. 5 and the results shown in FIG. 6 suggest that accumulation of comammox Nitrospira gradually progressed under the condition of pH 6 and was the dominant species on Day 120 of operation.

Further, at pH 5.0, a significant growth of comammox Nitrospira was observed in comparison to that at pH 5.5.

From these results, it has been found that use of nitrifying bacteria accumulated and cultured by the method in accordance with an embodiment of the present invention for culturing low temperature-resistant nitrifying bacteria makes it possible to achieve a good quality of treated water even under an acidic condition. The results have also suggested that an acidic environment is an advantageous condition for growth of comammox Nitrospira in a carrier.

### Example 6

In the present example, a device conducting nitrification at 5° C. for a long period of time was used to study involvement of comammox Nitrospira in low temperature nitrification.

As the device, the continuously operating device 102 illustrated in FIG. 4 was used. Inorganic synthetic wastewater used had a composition indicated in Table 6, and operation conditions employed are indicated in Table 7.

TABLE 6

| | Composition |
|---|---|
| Chemical name | (mg/L) |
| NH₄Cl | 150 |
| NaHCO₃ | 352 |
| Na₂HPO₄•12H₂O | 23 |

TABLE 7

| | |
|---|---|
| Inorganic | Composition: Table 6, NH₄—N: 40 mg/L, pH: 6 |
| synthetic | |
| wastewater | |
| Residence | 3 h-6 h |
| time of | |
| inorganic | |
| synthetic | |
| wastewater | |
| Water | 5° C.-6° C. |
| temperature | |
| Proportion of | Entrapping immobilization carrier (3 mm square) |
| carrier filling | was used in an amount of 15% by volume relative |
| | to volume of suspended sludge in inorganic |
| | synthetic wastewater |
| Aeration | 1 L/min |
| amount | |

The inorganic synthetic wastewater indicated in Table 6 was introduced into the continuously operating device 102, and a carrier was introduced as indicated in Table 7. A pH of the inorganic synthetic wastewater was adjusted to 6 by blowing air from an aeration device into the inorganic synthetic wastewater, and the time when the pH of the inorganic synthetic wastewater became 6 was regarded as Day 0 of operation.

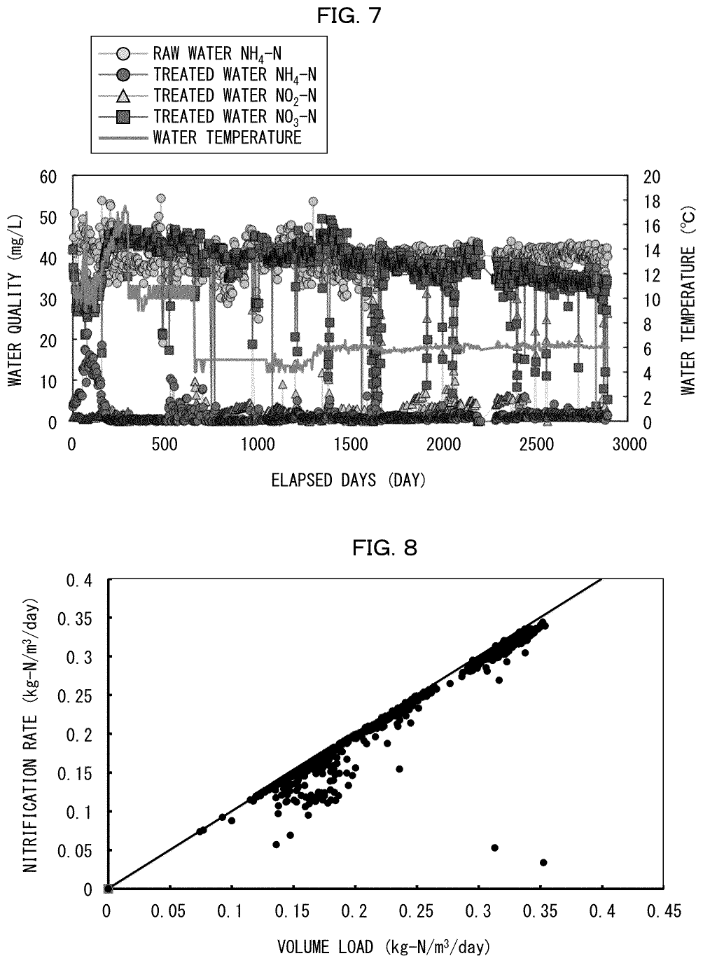

FIG. 7 is a view illustrating a result of checking a change over time in quality of the inorganic synthetic wastewater from a start to Day 2892 of operation. A horizontal axis in FIG. 7 represents the number of days that have passed since the start of the operation, and a vertical axis represents a concentration of nitrogen shown in an explanatory note. Concentrations of “RAW WATER NH₄—N”, “TREATED WATER NH₄—N”, “TREATED WATER NO₂—N”, and “TREATED WATER NO₃—N” were measured by the same method as the one described in Example 1.

It is indicated from FIG. 7 that the concentration of TREATED NO₃—N WATER was stably high and the concentrations of TREATED WATER NH₄—N and TREATED WATER NO₂—N were stably low. It is thus indicated that adjustment of the pH of the inorganic synthetic wastewater to 6 enabled stable nitrification even at a water temperature of 5° C. to 6° C.

FIG. 8 is a view illustrating a relationship between a volume load and a nitrification rate in the device illustrated in FIG. 4. A maximum nitrification rate of 0.34 kg-N/m³/day was achieved, and a nitrification ratio of substantially 100% was exhibited.

On Day 2779 of operation, a bacterial colony was collected from the carrier and subjected to real-time PCR. As a result, the entire eubacteria were 5.85×10¹⁰ copies/g of the carrier, ammonia-oxidizing bacteria were not more than a detection limit, and comammox Nitrospira was 7.49×10⁶ copies/g of the carrier. The real-time PCR was carried out by a method identical to the method described in Example 1, and the primers indicated in Table 1 were used. This result suggests that due to comammox Nitrospira being the dominant species, stable nitrification was carried out even at a water temperature of 5° C. to 6° C.

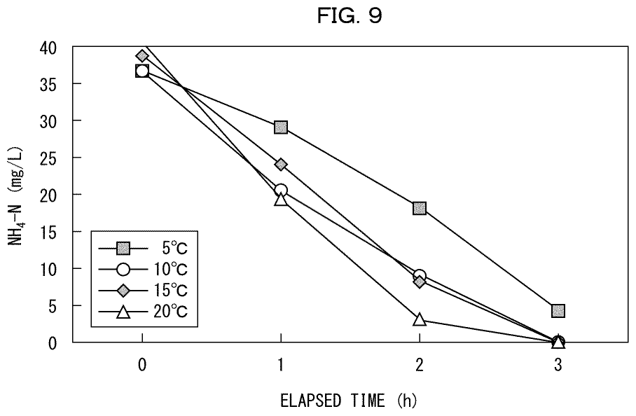

A batch treatment was carried out with a varying temperature of the nitrogen-containing water of 5° C., 10° C., 15° C., and 20° C. FIG. 9 is a view illustrating a result of a batch test. The result indicates that stable nitrification was possible at all of these temperatures.

From the above results, it is indicated that adjusting the pH of the inorganic synthetic wastewater to 6 makes it possible to cause the dominant species in the inorganic synthetic wastewater to be comammox Nitrospira. It is also indicated that use of a bacterial colony in which comammox Nitrospira has become the dominant species enables stable nitrification at a wide range of temperatures from 5° C. to 20° C.

### INDUSTRIAL APPLICABILITY

The present invention is effective for use in a sewage treatment plant and the like in which it is necessary to carry out nitrification under a low-temperature condition.

### REFERENCE SIGNS LIST

1, 1′: Denitrification tank
2, 2′: Nitrification tank
3: Culture tank
4: Acid storage tank
5: Alkali storage tank
*1: Introduction of return sludge from nitrification tank 2 into culture tank 3
*2: Introduction of nitrification liquid from nitrification tank 2 into denitrification tank 1
6: Sedimentation tank
*a: Introduction of nitrification liquid from sedimentation tank 6 into denitrification tank 1′
*b: Introduction of return sludge from sedimentation tank 6′ into nitrification tank 2
100: Device for treating nitrogen-containing water
101: Device for treating nitrogen-containing water
A: Treatment system A
B: Treatment system B

1. A method for culturing low temperature-resistant nitrifying bacteria, the method comprising the step of:
adjusting a pH of nitrogen-containing water a containing ammonia nitrogen and/or organic nitrogen to not more than 6.0 to obtain nitrogen-containing water b in which a dominant species of microorganisms is low temperature-resistant nitrifying bacteria.

2. The method as set forth in claim 1, wherein the pH of the nitrogen-containing water a is adjusted to more than 3.0 and not more than 6.0.

3. The method as set forth in claim 1, wherein a concentration of the ammonia nitrogen and/or the organic nitrogen in the nitrogen-containing water a is not more than 2000 mg-N/L.

4. The method as set forth in claim 1, wherein a ratio of an amount of the low temperature-resistant nitrifying bacteria to an amount of ammonia-oxidizing bacteria is more than 1 in the nitrogen-containing water b.

5. The method as set forth in claim 4, wherein the ratio is not less than 5 and not more than 100.

6. The method as set forth in claim 1, wherein the low temperature-resistant nitrifying bacteria are comammox Nitrospira.

7. A method for treating nitrogen-containing water, the method comprising the step of:
nitrifying, at a water temperature of not lower than 0° C. and not higher than 15° C. in an aerobic atmosphere with use of low temperature-resistant nitrifying bacteria obtained by a method recited in claim 1, ammonia contained in the nitrogen-containing water a and/or the nitrogen-containing water b.

8. A device for treating nitrogen-containing water, the device comprising a culture tank and a nitrification tank, wherein:
the culture tank and the nitrification tank are capable of circulating content between each other;
the culture tank (i) is capable of storing therein (a) nitrogen-containing water a containing ammonia nitrogen and/or organic nitrogen and (b) return sludge obtained from the nitrification tank, and (ii) produces nitrogen-containing water b from the nitrogen-containing water a by adjusting a pH of the nitrogen-containing water a to not more than 6.0 and thereby causing a dominant species of microorganisms contained in the nitrogen-containing water a to be low temperature-resistant nitrifying bacteria; and
the nitrification tank (i) is capable of storing therein the nitrogen-containing water a and the nitrogen-containing water b and (ii) produces a nitrification liquid by nitrifying, at a water temperature of not lower than 0° C. and not higher than 15° C. with use of the low temperature-resistant nitrifying bacteria contained in the nitrogen-containing water b, ammonia contained in the nitrogen-containing water a and/or the nitrogen-containing water b.

9. A device for treating nitrogen-containing water, the device comprising a treatment system A and a treatment system B, wherein:
the treatment system A includes a nitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the nitrification liquid obtained in the nitrification tank I, and a nitrification liquid obtained in the nitrification tank II, (ii) denitrifies nitric acid contained in each of the nitrification liquid obtained in the nitrification tank I and the nitrification liquid obtained in the nitrification tank II, and (iii) transfers the nitrogen-containing water a to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. in an aerobic atmosphere with use of the low temperature-resistant nitrifying bacteria, and (iii) transfers the nitrification liquid to the denitrification tank II.

10. A device for treating nitrogen-containing water, the device comprising a treatment system A and a treatment system B, wherein:
the treatment system A includes a nitrification tank I and a denitrification tank I;
the nitrification tank I (i) is capable of storing therein (a) nitrogen-containing water a containing ammonia nitrogen and/or organic nitrogen and (b) return sludge from the treatment system B, (ii) adjusts a pH of the nitrogen-containing water a to not more than 6.0 to cause a dominant species of microorganisms contained in the nitrogen-containing water a to be low temperature-resistant nitrifying bacteria, and (iii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge;
the denitrification tank I denitrifies nitric acid contained in the nitrification liquid obtained in the nitrification tank I and transfers obtained return sludge to the treatment system B;
the treatment system B includes a nitrification tank II and a denitrification tank II;
the nitrification tank II and the denitrification tank II are capable of circulating content between each other;
the denitrification tank II (i) is capable of storing therein nitrogen-containing water a containing ammonia nitrogen and/or organic nitrogen, the return sludge from the denitrification tank I, and a nitrification liquid obtained in the nitrification tank II, (ii) denitrifies nitric acid contained in the nitrification liquid, and (iii) transfers the nitrogen-containing water a and the return sludge to the nitrification tank II; and
the nitrification tank II (i) adjusts a pH of the nitrogen-containing water a transferred from the denitrification tank II to not more than 6.0 to cause a dominant species of microorganisms contained in the nitrogen-containing water a to be low temperature-resistant nitrifying bacteria, (ii) produces a nitrification liquid by carrying out nitrification at a temperature of not lower than 0° C. and not higher than 15° C. with use of the low temperature-resistant nitrifying bacteria and low temperature-resistant nitrifying bacteria contained in the return sludge, and (iii) transfers the nitrification liquid to the denitrification tank II.


SEQUENCE LISTING (9 sequences)

SEQ ID NO: 1 - DNA, 18 bp, synthetic construct
  source 1..18: mol_type = other DNA
ggggtttctactggtggt

SEQ ID NO: 2 - DNA, 21 bp, synthetic construct
  source 1..21: mol_type = other DNA
cccctcbgsaaavccttcttc

SEQ ID NO: 3 - DNA, 19 bp, synthetic construct
  source 1..19: mol_type = other DNA
ctgaytgggcytggacatc

SEQ ID NO: 4 - DNA, 20 bp, synthetic construct
  source 1..20: mol_type = other DNA
ttcttctttgttgcccagta

SEQ ID NO: 5 - DNA, 20 bp, synthetic construct
  source 1..20: mol_type = other DNA
ggatttctggntsgattgga

SEQ ID NO: 6 - DNA, 20 bp, synthetic construct
  source 1..20: mol_type = other DNA
wagttngaccaccastacca

SEQ ID NO: 7 - DNA, 18 bp, synthetic construct
  source 1..18: mol_type = other DNA
cggtgaatacgttcycgg

SEQ ID NO: 8 - DNA, 19 bp, synthetic construct
  source 1..19: mol_type = other DNA
cgwtaccttgttacgactt

SEQ ID NO: 9 - DNA, 19 bp, synthetic construct
  source 1..19: mol_type = other DNA
cttgtacacaccgcccgtc